(19) United States
(12) Patent Application Publication
(10) Pub. No.: US 2024/0158813 A1
(43) Pub. Date: May 16, 2024

(54) MODULATION OF NANOG

(71) Applicant: Leonhardt Ventures LLC, Mission Viejo, CA (US)

(72) Inventors: Howard J. Leonhardt, Mission Viejo, CA (US); Jorge Genovese, Irvine, CA (US)

(21) Appl. No.: 18/504,918

(22) Filed: Nov. 8, 2023

Related U.S. Application Data
(60) Provisional application No. 63/383,218, filed on Nov. 10, 2022.

(51) Int. Cl. C12N 15/87; A61N 1/36; C12N 13/00

(52) U.S. Cl. CPC C12N 15/87; A61N 1/36121; C12N 13/00

(57) ABSTRACT

Described is a low voltage, pulsed electrical stimulation device for modulating the expression of NANOG, a useful protein, by cells and tissues. Also described are methods of enhancing expression of NANOG in cells, particularly a method of stimulating the expression and/or release of NANOG in a cell having a gene encoding NANOG, wherein the method includes applying a bioelectric signal of, within 15%, 1 mA to about 5 mA, 20 pulses per second, at 400 microsecond pulse duration to the cell (e.g., directly, indirectly, or wirelessly).

### CROSS-REFERENCE TO RELATED APPLICATION

This application claims the benefit under 35 U.S.C. § 119(e) of U.S. Provisional Patent Application Ser. No. 63/383,218, filed Nov. 10, 2022, the disclosure of which is hereby incorporated herein in its entirety by this reference.

### TECHNICAL FIELD

The application relates generally to the field of medical devices and associated methods of treatment, and more specifically to methods of treatment involving the precise bioelectrical stimulation of a subject's tissue, optionally augmented with the administration of a composition comprising, among other things, stem cells and nutrients, useful to increase the expression and/or release of NANOG to stimulate and treat the subject, the subject's tissue(s), the subject's organ(s), and/or the subject's cells. More specifically, the application relates to a device programmed to produce bioelectric signals, and associated methods for the controlled expression of secreted NANOG via application of such bioelectric signals.

### BACKGROUND

NANOG is a transcription factor in embryonic stem cells (“ESCs”) and is thought to be a key factor in maintaining pluripotency. ESCs offer an important area of study because of their ability to maintain pluripotency. In other words, these cells have the ability to become virtually any cell of any of the three germ layers (endoderm, ectoderm, and mesoderm).

As found by Shahini et al., “Overexpression of the transcription factor NANOG in senescent myoblasts can overcome the effects of cellular senescence and confer a youthful phenotype to senescent cells. NANOG ameliorated primary hallmarks of cellular senescence including genomic instability, loss of proteostasis, and mitochondrial dysfunction. The rejuvenating effects of NANOG included restoration of DNA damage response via up-regulation of DNA repair proteins, recovery of heterochromatin marks via up-regulation of histones, and reactivation of autophagy and mitochondrial energetics via up-regulation of AMP-activated protein kinase (AMPK). Expression of NANOG in the skeletal muscle of a mouse model of premature aging restored the number of myogenic progenitors and induced formation of eMyHC+ myofibers.” Shahini et al., “Ameliorating the hallmarks of cellular senescence in skeletal muscle myogenic progenitors in vitro and in vivo,” Science Advances vol. 7, no. 36 (3 September 2021). See, also, J. van Zijl, “Scientists Reverse the Aging of Skeletal Muscle in Longevity Breakthrough” iflscience.com/scientists-reverse-the-aging-of-skeletal-muscle-in-longevity-breakthrough-65380 (Sep. 19, 2022).

Similarly, Park et al. (2019), infra, found that overexpression of NANOG in amniotic fluid-derived mesenchymal stem cells accelerates dermal papilla cell activity and promotes hair follicle regeneration.

There have even been suggestions that NANOG can reverse aging. See, e.g., Mistriotis et al. (2017), infra, and C. Nealon-Buffalo (2016), infra.

It would be an improvement in the art to have a way to modulate the expression of NANOG.

### BRIEF SUMMARY

Described herein is a bioelectric stimulator particularly configured to modulate (e.g., upregulate or downregulate) the expression and/or release of NANOG in cellular tissue.

Further described is a precise bioelectric signal that upregulate levels of circulating NANOG on demand with control.

Particularly described is a bioelectric stimulator comprising an electric signal generator and electrode(s), which electric signal generator is programmed to produce at least one bioelectric signal that stimulates target tissue comprising living cells so as to modulate expression and/or release of NANOG by the living cells of the target tissue, wherein the bioelectric signal comprises, within 15%, a bioelectric signal having a biphasic pulse at a frequency of, within 15%, 20 Hz, with a pulse duration/width of 400 microseconds (μsec) and has a current of 1 milliAmp (mA) to 5 mA as may be measured at the level of the living cells of the target tissue.

Also described are methods of using such a bioelectric stimulator to stimulate target tissue comprising living cells of a subject to modulate the expression of NANOG by the living cells, the method comprising: administering the bioelectric signal to the living cells via the electrode(s) for from about 5 minutes to about an hour, so as to modulate the expression of NANOG by the living cells.

Such a method may be used to treat a subject, such as one diagnosed as suffering from an aging-related ailment, such as heart failure, erectile dysfunction, osteopenia, osteoporosis, metabolic stress, metabolic syndrome, skin disorder(s), hair loss, sexual dysfunction, energy homeostasis, lack of physical activity, diabetes, hypertension, arterial calcification, valve calcification, Alzheimer's disease, dementia, cognitive function, depression, addiction, pre-mature aging-related disorder, senescence, aging, muscle atrophy, and/or inflammation. NANOG may not only have the capacity to delay aging, quite remarkably, it may even have the potential in some cases to reverse aging, especially muscle related aging, including the heart muscle. Studies have shown cells with increased NANOG expression also seem to exhibit superior DNA damage repair. NANOG has a number of properties potentially useful for heart muscle regeneration including the ability to restore myogenic differentiation potential of skeletal myoblasts. NANOG has been shown to reverse aging of muscle tissue.

Also described is a method of inhibiting expression of NANOG in a subject utilizing a bioelectric stimulator, wherein the bioelectric stimulator comprises an electric signal generator and associated electrode(s), which electric signal generator is programmed to produce at least one bioelectric signal that stimulates target tissue comprising living cells so as to inhibit expression and/or release of NANOG by the living cells of the target tissue. Such methods may be used, for example, to downregulate expression of NANOG in a subject after stimulation, e.g., as described herein or for treating or preventing cancer.

### BRIEF DESCRIPTION OF THE DRAWINGS

FIG. 1 depicts a programmed bioelectric stimulator for delivery to a subject connected to multiple soft conductive electrode pads.

FIG. 2 depicts a programmed bioelectric stimulator as described herein.

FIG. 3 depicts a conductive soft wrap which may be used with the described system.

FIG. 4 depicts a programmed bioelectric stimulator depicted alongside a pen.

FIG. 5 depicts a bioelectric stimulation system for laboratory testing.

### DETAILED DESCRIPTION

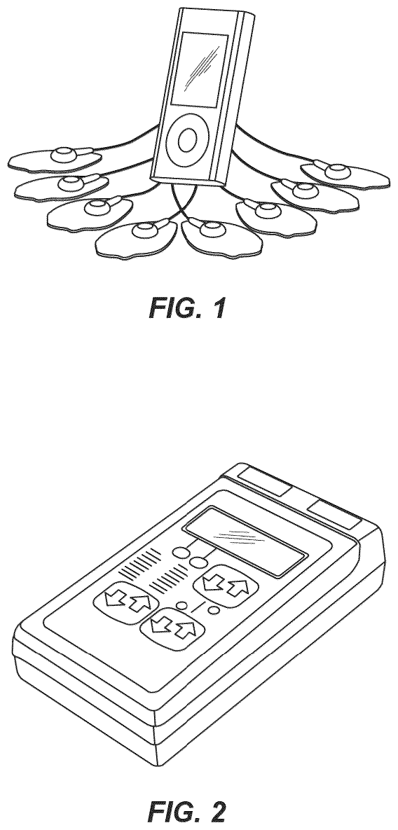

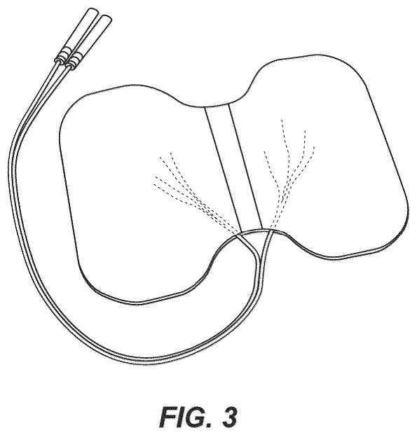

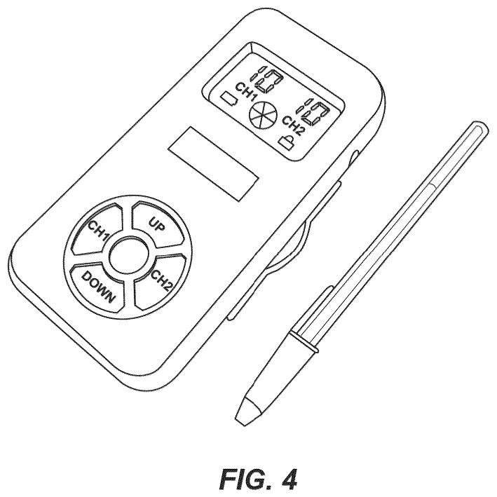

Referring now to FIG. 1, depicted is a stimulator for use in treating a human. The depicted device is about the size of a pen (FIG. 4) and is programmable.

A micro voltage signal generator for use herein may be produced utilizing the same techniques to produce a standard heart pacemaker well known to a person of ordinary skill in the art. An exemplary microvoltage generator is available from Mettler Electronics Corp. of Anaheim, California, US or HTM Electronica of Amparo, BR. The leading pacemaker manufacturers are Medtronic, Boston Scientific Guidant, Abbott St. Jude, BioTronik and Sorin Biomedica.

Construction of the electric signal generators and pacemakers are known in the art and can be obtained from OEM suppliers as well as their accompanying chargers and programmers. The electric signal generators are programmed to produce specific bioelectric signals to lead to specific protein expressions at precisely the right time for, e.g., optimal treatment or for tissue regeneration.

The biostimulator of FIG. 1 is depicted with multiple soft conductive electrode pads. Electrodes may be used to deliver a bioelectric signal to the subject by applying the electrodes to the subject's skin (e.g., on the skin above the thigh muscles or on the skin above the kidneys). In certain embodiments, a bioelectric stimulator is in electrical connection with a conductive soft wrap.

A bench top stimulator (e.g., a Mettler Model 240 Stimulator from Mettler Electronics of Anaheim, CA, US) may be pre-programmed with the bioelectric signaling sequence(s) for controlling the expression and/or release of NANOG.

In some embodiments, the application of bioelectric signals can further be used to modulate (e.g., upregulate) expression by the subject's cells the production of other biological molecules in addition to NANOG. See, e.g., U.S. Pat. No. 10,960,206 to Leonhardt et al. (Mar. 30, 2021) for “Bioelectric Stimulator,” the contents of which are incorporated herein by this reference.

For example, in certain embodiments (e.g., in order to provide antiaging effects, cognitive improvements, sexual health, and/or cardiovascular protection), further specific bioelectric signals are applied that upregulate expression of NANOG, klotho, and sirtuin.

Further, in certain embodiments, bioelectric controlled modulating (e.g., upregulating) expression of the NANOG family of proteins is added to bioelectric signals for modulating expression and/or release of klotho, follistatin, tropoelastin, stromal cell-derived factor-1 (“SDF1”) (a stem cell recruiting signal), platelet-derived growth factor (“PDGF”), and/or insulin-like growth factor 1 (“IGF-1”) for use, for example, in anti-aging therapy and muscle atrophy recovery. See, e.g., the incorporated U.S. Pat. No. 10,960,206 to Leonhardt et al. for such bioelectric signals.

The treatment and/or prevention of depression may involve the application of further bioelectric signal(s) to upregulate expression of Klotho. See, e.g., U.S. Patent Application Publication US 2020-0289826-A1 to Leonhardt et al. (Sep. 17, 2020) for “Klotho Modulation” and U.S. Patent Application Publication US 20210402184-A1 to Leonhardt et al. (Dec. 31, 2021) for “Klotho Modulation.”

An implantable medical lead is described in U.S. Pat. No. 8,442,653 to Gill (May 14, 2013) for “Brain Electrode,” the contents of which are incorporated herein by this reference.

In, for example, FIG. 15 of the incorporated U.S. Pat. No. 10,960,206 to Leonhardt et al., depicts an image of the signal (voltage and frequency) associated with stem cell proliferation: 2.5-6.0 V (4V depicted there), 20 Hz, pulse width 200-700 μsec, square wave. No special effects are attributed to a pulse width of 400 μsec, and NANOG is not mentioned.

Both wireless non-invasive and/or implantable wire lead (“electrode”) based means may be used to deliver the regeneration and healing promoting bioelectric signal(s) to target organs such as the brain.

A wireless, single lumen infusion pacing lead or infusion conduction wide array patch may all be used to deliver the regeneration signals and substances to the organ of interest to be treated or they may be used in combination.

A re-charging wand for use herein is preferably similar to the pacemaker re-charging wand developed by Alfred Mann in the early 1970's for recharging externally implantable pacemakers.

Bioelectric stimulation can be done with the described bioelectric stimulator, which can have a pacing infusion lead with, e.g., a corkscrew lead placed/attached at, e.g., the center of the tissue to be stimulated and/or treated.

The bioelectric stimulator is actuated and runs through programmed signals to signal the release of, e.g., NANOG. In such a method, the electrical signal may be measured three (3) mm deep into the tissue.

In certain embodiments, an increase in upregulation of expression of NANOG by over three times was shown utilizing specific bioelectric signaling sequences. Further, in certain embodiments, down regulation of NANOG was also three times.

Relationship Between the Components:

The micro voltage signal generator is attached to the pacing infusion lead with, e.g., a brain electrode (Medtronic) (e.g., for bioelectric stimulation of the brain), or conductive polymer bandage or patch to the tissue or organ to be treated. An external signal programmer may be used to program the micro voltage signal generator with the proper signals for treatment including the NANOG producing signal(s). The device battery may be re-chargeable with an external battery charging wand.

The essential elements are the micro voltage signal generator and the means for delivering the signal to the target tissue.

The signal generator may be external or internal. The transmission of the signal may be wireless, via liquid and/or via wires.

The tissue contact interface may be, e.g., a patch or bandage or may be via electrodes or leads. FDA cleared gel tape electrodes (Mettler) may be used for skin delivery. Electro acupuncture needles may be used to ensure the signals positively reach target tissues under the skin.

In certain preferred embodiments a method of stimulating the expression of NANOG in a living cell having a gene encoding a NANOG is described, wherein the method comprises: applying to the cell a bioelectric signal of, within 15%, 20 Hz at, within 15%, 400 μsec pulse width duration, wherein the amount of NANOG expression enhanced by this bioelectric signal is greater than that seen with a generic bioelectric cell stimulation alone as may be determined by an analysis of the upregulation of mRNA level/GAPDH fold gene expression in the cell in each situation.

NANOG activity in treating disease states and conditions is many fold.

The invention is further described by the following illustrative Examples.

### EXAMPLES

### Example I

Upregulation of Expression of NANOG

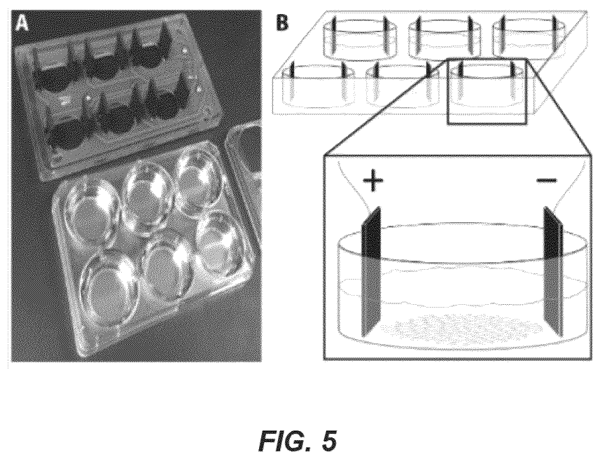

FIG. 5 depicts a bioelectric stimulation system. Cells and/or tissue are plated in each dish and cultured. Stimulation occurs using an electrode array (shown at the top of panel A), which is inverted and introduced into the 6-well dish where cells are grown. Each well receives uniform stimulation via a pair of carbon electrodes.

Various currents were applied to human fibroblasts including 1 mA and 5 mA. In each stimulation, 20 pps frequency (as expressed by Mettler electric signal generators) and 400 μs pulse duration for 60 minutes were applied to human fibroblasts.

Applying a bioelectric signal of 1 mA, 20 pps, 400 μs pulse duration, to living human fibroblast cells for 1 hour resulted in a 44% increase in NANOG expression over control.

Applying a bioelectric signal of 5 mA, 20 pps, 400 μs pulse duration, to living human fibroblast cells for 1 hour resulted in a 23% increase in NANOG expression over control.

### Example II

The electrodes of a bioelectric stimulator built and programmed as described herein is utilized to apply a bioelectric signal of 1 mA, 20 pps, 400 μs pulse duration, to target tissue of a subject for from five minutes to 1 hour. NANOG expression is upregulated in cells contained within the target tissue thereby.

### REFERENCES

(The contents of the entirety of each of which is incorporated herein by this reference.)
Mistriotis et al., “NANOG Reverses the Myogenic Differentiation Potential of Senescent Stem Cells by Restoring ACTIN Filamentous Organization and SRF-Dependent Gene Expression,” Stem Cells, Volume 35, Issue 1, January 2017, Pages 207-221, doi.org/10.1002/stem.2452.
C. Nealon-Buffalo, “Can a Stem Cell Gene Called NANOG Reverse Aging?” (Jul. 27, 2016); www.futurity.org/nanog-aging-stem-cells-1212112-2/.
Park et al., “Overexpression of Nanog in amniotic fluid-derived mesenchymal stem cells accelerates dermal papilla cell activity and promotes hair follicle regeneration,” Exp. Mol. Med. 51(7):1-15 (July 2019); doi: 10.1038/s12276-019-0266-7. PMID: 31273189; PMCID: PMC6802618.
Shahini et al., “Ameliorating the hallmarks of cellular senescence in skeletal muscle myogenic progenitors in vitro and in vivo,” Science Advances vol. 7, no. 36 (3 Sep. 2021); DOI: 10.1126/sciadv.abe567.
J. van Zijl, “Scientists Reverse the Aging of Skeletal Muscle in Longevity Breakthrough,” iflscience.com/scientists-reverse-the-aging-of-skeletal-muscle-in-longevity-breakthrough-65380 (Sep. 19, 2022).
U.S. Pat. No. 8,442,653 to Gill (May 14, 2013) for “Brain Electrode.”
U.S. Patent Application Publication US 2020-0289826-A1 to Leonhardt et al. (Sep. 17, 2020) for “Klotho Modulation.”
U.S. Patent Application Publication US 2021-0402184-A1 to Leonhardt et al. (Dec. 31, 2021) for “Klotho Modulation.”
U.S. Pat. No. 10,960,206 to Leonhardt et al. (Mar. 30, 2021) for “Bioelectric Stimulator.”

What is claimed is:

1. A method of using a bioelectric stimulator comprising an electric signal generator and electrode(s) to stimulate living cells to modulate the expression of NANOG by the cells,
wherein the electric signal generator is programmed to produce at least one bioelectric signal that stimulates the cells to upregulate expression and/or release of NANOG by the cells, said at least one bioelectric signal having a biphasic pulse at a frequency of, within 15%, 20 Hz, with a pulse width of, within 15%, and 400 microseconds (μsec), the method comprising:
administering the bioelectric signal to the cells via the electrode(s) so as to modulate the expression of NANOG by the cells.

2. The method according to claim 1, wherein the bioelectric signal has a current of from 1 mA to 5 mA as may be measured at the level of the stimulated cells.

3. The method according to claim 1, wherein the bioelectric signal is administered to the cells via the electrode(s) for from about five (5) minutes to about an hour.

4. The method according to claim 2, wherein the bioelectric signal is administered to the cells via the electrode(s) for from about five (5) minutes to about an hour.

5. The method according to claim 1, wherein the cells are comprised within target tissue of a subject.

6. The method according to claim 2, wherein the cells are comprised within target tissue of a subject.

7. The method according to claim 3, wherein the cells are comprised within target tissue of a subject.

8. The method according to claim 4, wherein the cells are comprised within target tissue of a subject.

9. The method according to claim 5, wherein the subject is suffering from heart failure, erectile dysfunction, osteopenia, osteoporosis, metabolic stress, metabolic syndrome, spinal cord injury, liver disease or dysfunction, skin disorder(s), hair loss, sexual dysfunction, energy homeostasis, lack of physical activity, diabetes, hypertension, arterial calcification, valve calcification, Alzheimer's disease, dementia, cognitive function, depression, addiction, pre-mature aging related disorder, senescence, aging, muscle atrophy, and/or inflammation.

10. The method according to claim 6, wherein the subject is suffering from heart failure, erectile dysfunction, osteopenia, osteoporosis, metabolic stress, metabolic syndrome, spinal cord injury, liver disease or dysfunction, skin disorder(s), hair loss, sexual dysfunction, energy homeostasis, lack of physical activity, diabetes, hypertension, arterial calcification, valve calcification, Alzheimer's disease, dementia, cognitive function, depression, addiction, pre-mature aging related disorder, senescence, aging, muscle atrophy, and/or inflammation.

11. The method according to claim 7, wherein the subject is suffering from heart failure, erectile dysfunction, osteopenia, osteoporosis, metabolic stress, metabolic syndrome, spinal cord injury, liver disease or dysfunction, skin disorder(s), hair loss, sexual dysfunction, energy homeostasis, lack of physical activity, diabetes, hypertension, arterial calcification, valve calcification, Alzheimer's disease, dementia, cognitive function, depression, addiction, pre-mature aging related disorder, senescence, aging, muscle atrophy, and/or inflammation.

12. The method according to claim 8, wherein the subject is suffering from heart failure, erectile dysfunction, osteopenia, osteoporosis, metabolic stress, metabolic syndrome, spinal cord injury, liver disease or dysfunction, skin disorder(s), hair loss, sexual dysfunction, energy homeostasis, lack of physical activity, diabetes, hypertension, arterial calcification, valve calcification, Alzheimer's disease, dementia, cognitive function, depression, addiction, pre-mature aging related disorder, senescence, aging, muscle atrophy, and/or inflammation.

13. A method of stimulating a living cell to modulate expression and/or release of NANOG by the cell, the method comprising:
administering at least one bioelectric signal to the cell so as to upregulate expression and/or release of NANOG by the cell,
wherein the at least one bioelectric signal has a biphasic pulse at a frequency of, within 15%, 20 Hz, with a pulse width of, within 15%, and 400 microseconds (μsec).

14. The method according to claim 13, wherein the bioelectric signal has a current of from 1 mA to 5 mA as may be measured at the level of the cell.

15. The method according to claim 13, wherein the bioelectric signal is administered to the cell for from about five (5) minutes to about an hour.

16. The method according to claim 14, wherein the bioelectric signal is administered to the cell for from about five (5) minutes to about an hour.

17. The method according to claim 13, wherein the cell is comprised within target tissue of a subject.

18. The method according to claim 14, wherein the cell is comprised within target tissue of a subject.

19. The method according to claim 15, wherein the cell is comprised within target tissue of a subject.

20. The method according to claim 16, wherein the cell is comprised within target tissue of a subject.
